(19) United States
(12) Patent Application Publication
(10) Pub. No.: US 2021/0093104 A1
(43) Pub. Date: Apr. 1, 2021

(54) Weighted Blanket with Cooling Gel Filling

(71) Applicant: Craig W. Clark, Denver, CO (US)

(72) Inventor: Craig W. Clark, Denver, CO (US)

(21) Appl. No.: 16/586,784

(22) Filed: Sep. 27, 2019

(51) Int. Cl. A47G 9/02

(52) U.S. Cl. CPC A47G 9/0215; A47G 2009/008; A47G 9/0223

(57) ABSTRACT

The current invention is a cooling gel blanket that is filled by weighted material and with materials to disperse heat. The cooling gel blanket is made by laminating a polyester or cotton fabric over a gel cooling core. The cooling gel is a water based polyurethane. The inner blanket core is a flexible cooling gel that transfers heat from the body to the room acting as a heat sink.

### CROSS-REFERENCES TO RELATED APPLICATIONS (IF ANY)

None.

### BACKGROUND

### 1. Field of the Invention

This invention relates to blankets and particularly ones that have gel cooling layers.

### 2. Description of Prior Art

Blankets are comforting but can get people too hot especially in the summer months. There needs to be an easy way for blankets to cool as well as providing warmth.

There is still room for improvement in the art.

### SUMMARY OF THE INVENTION

The current invention is a cooling gel blanket that is filled by weighted material and with materials to disperse heat.

The cooling gel blanket is made by laminating a polyester or cotton fabric over a gel cooling core. The inner blanket core is a flexible cooling gel that transfers heat from the body to the room acting as a heat sink.

The thickness of the cooling blanket is approximately 1 mm. The blanket has duvet loops sewn into the corners to attach a duvet. The edge is also visible in this photo which shows how the fabric is overlapped and sewn together creating a finished edge.

### BRIEF DESCRIPTION OF THE DRAWINGS

Without restricting the full scope of this invention, the preferred form of this invention is illustrated in the following drawings:

FIG. 1 shows the blanket from the top front, as it would appear on a bed;

FIG. 2 shows the top of the blanket; folded on itself, showing the top of the blanket, bottom of the blanket, and the stitching;

FIG. 3 shows the bottom of the blanket;

FIG. 4 shows the left side of the blanket;

FIG. 5 shows the right side of the blanket;

FIG. 6 shows the top of the blanket;

FIG. 7 shows the bottom of the blanket;

FIG. 8 shows the cooling gel packets; and

FIG. 9 shows a cross section of the blanket and the cooling gel packets;

FIG. 10 shows a cross section of the blanket where the cooling gel packets are attached;

FIG. 11 shows a cross section of the blanket where there is a cooling gel layer.

### DESCRIPTION OF THE PREFERRED EMBODIMENT

There are a number of significant design features and improvements incorporated within the invention.

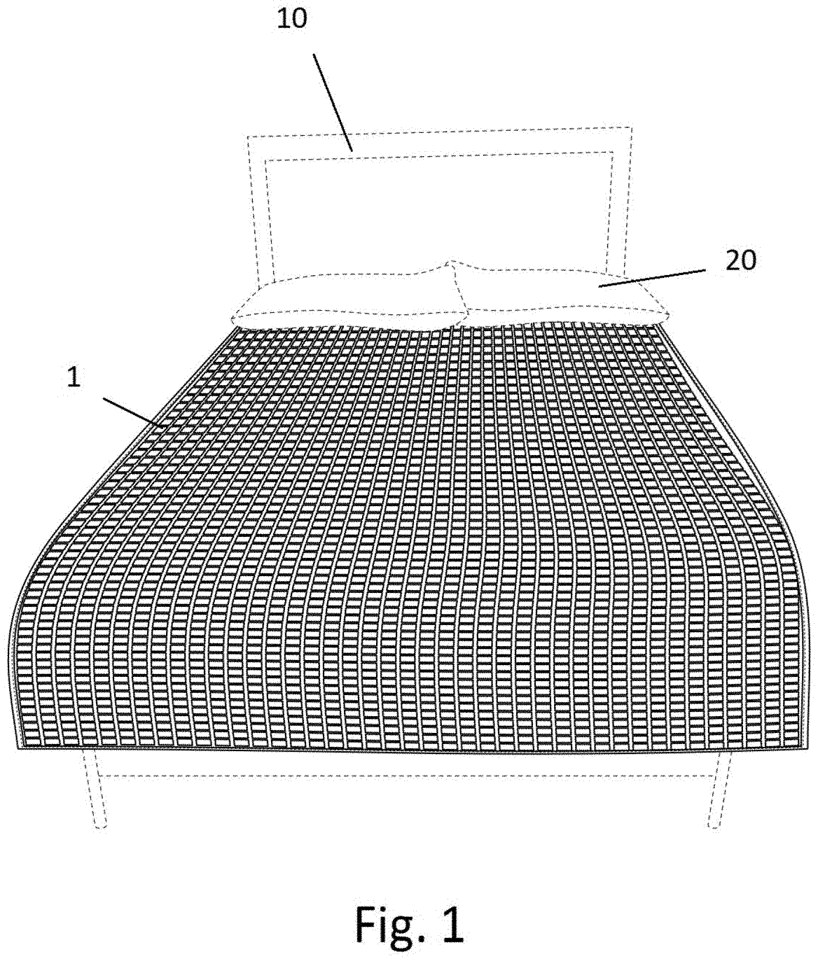

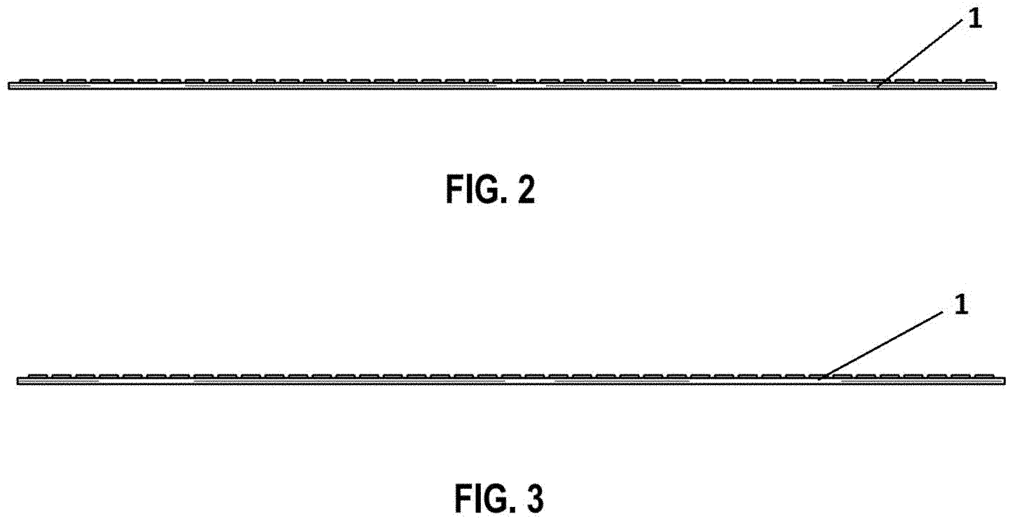

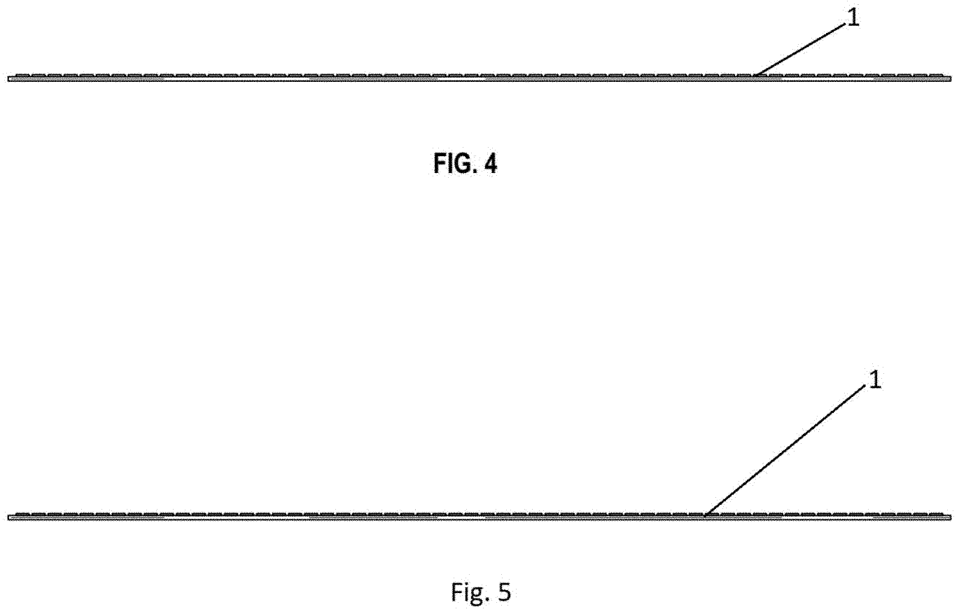

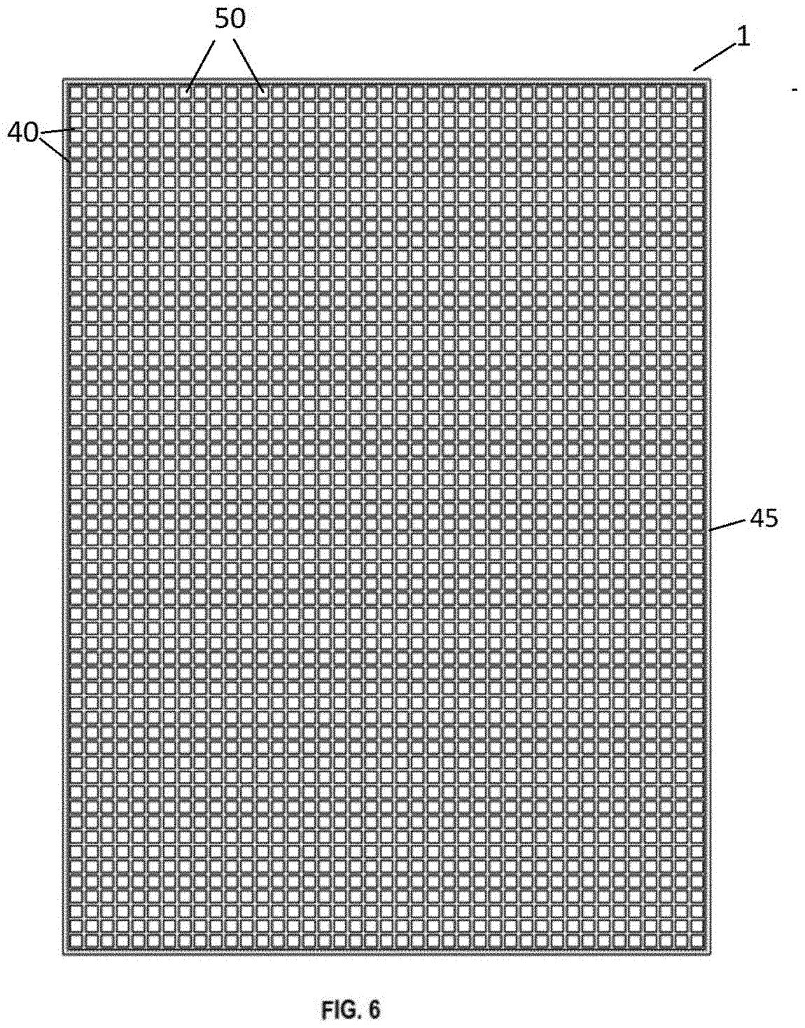

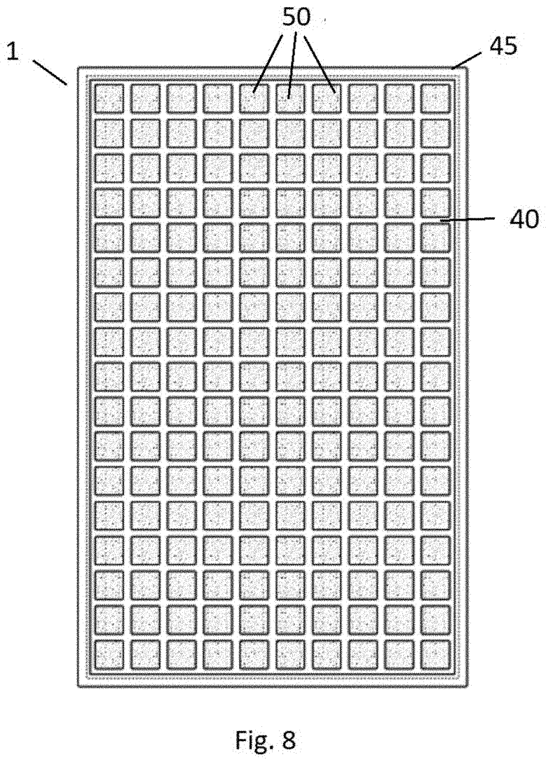

The current invention is a cooling gel blanket 1 that is filled by weighted material and with materials to disperse heat as shown in FIGS. 1 through 11. As shown in FIG. 1, the cooling gel blanket 1 can be placed on a bed 10 and can be used with pillows 20.

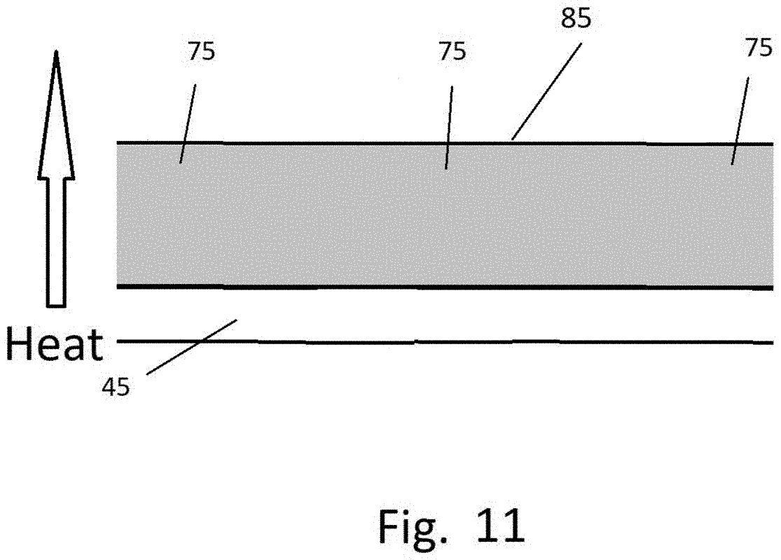

The cooling gel blanket 1 is made by laminating a polyester or cotton fabric 45 with a cooling gel 75. The cooling blanket 1 has a flexible cooling gel 75 that transfers heat from the body to the room acting as a heat sink as shown in FIG. 11.

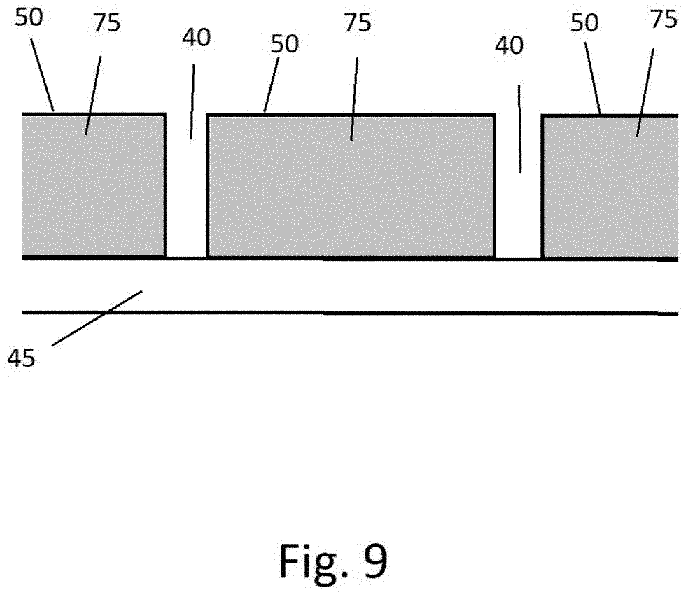

The cooling gel blanket is made by laminating a polyester or cotton fabric 45 over a gel cooling core 50 as shown in FIG. 9.

The inner blanket gel cooling core 50 is a flexible cooling gel that transfers heat from the body to the room acting as a heat sink. The cooling gel 75 is a water based polyurethane.

In the preferred embodiment, the top of the cooling blanket 1 will have a layer of rectangular packets 50 the contain the cooling gel 75 where the packets 50 are divided by evenly spaced gaps 40 as shown in FIG. 9.

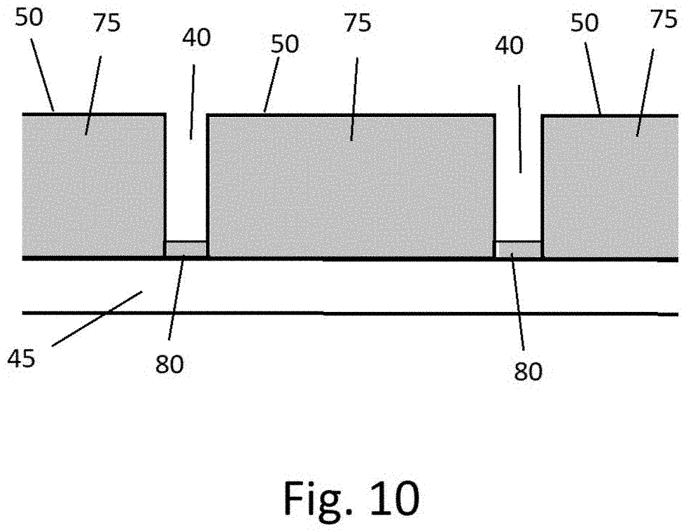

In another embodiment, the packets 50 are connected by connectors 80 that allow the cooling gel 75 to flow evenly between the packets 50 as shown in FIG. 10.

In another embodiment, the cooling gel 75 is contained in a cooling gel layer 85 as shown in FIG. 11.

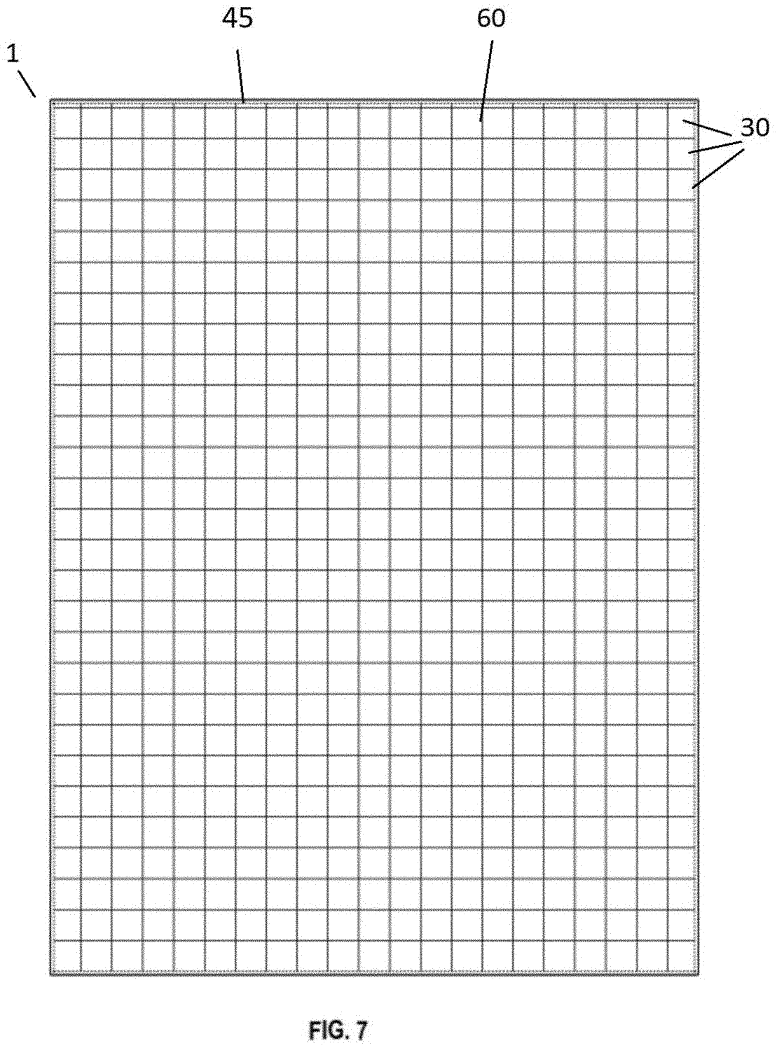

The back of the cooling gel blanket 1, as shown in FIG. 7, shows the threaded pattern 60 of squares 30 used to connect and hold the layers of the cooling gel blanket together with the cooling gel 75 laminated onto the polyester or cotton fabric 45.

### Advantages

The current invention is the only weighted blanket with a cooling gel allowing for confirmable sleep.

Although the present invention has been described in considerable detail with reference to certain preferred versions thereof, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided. With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

That which is claimed is:

1. A device comprising:
a blanket is made by laminating a fabric over a cooling gel.

2. The device according to claim 1 further comprising:
where the cooling gel is a water based polyurethane.

3. The device according to claim 1 further comprising:
where the cooling gel is flexible.

4. The device according to claim 1 further comprising:
where the cooling gel pulls heat away from a body.

5. The device according to claim 1 further comprising:
where the cooling gel is flexible.

6. The device according to claim 1 further comprising:
where the cooling gel laminated onto polyester or cotton fabric.

7. The device according to claim 1 further comprising:
having a layer of rectangular packets that contain the cooling gel where the packets are divided by evenly spaced gaps

8. The device according to claim 7 further comprising:
where the packets are connected.
